(19) United States
(12) Patent Application Publication
(10) Pub. No.: US 2017/0035162 A1
(43) Pub. Date: Feb. 9, 2017

(54) FRAGRANCE VOLATIZING JEWELRY AND METHODS OF PRODUCING THE SAME

(71) Applicant: Adoram Leshem, Tel-Aviv (IL)

(72) Inventor: Adoram Leshem, Tel-Aviv (IL)

(21) Appl. No.: 15/202,318

(22) Filed: Jul. 5, 2016

(30) Foreign Application Priority Data
Aug. 3, 2015 (GB) 1513744.1

(51) Int. Cl. A44C 15/00; A44C 5/00; A61L 9/12; A44B 15/00; A45D 34/00; A44C 25/00; A44C 27/00

(52) U.S. Cl. CPC A44C 15/002; A44C 25/001; A44C 5/0007; A44C 27/00; A44B 15/005; A45D 34/00; A61L 9/12; A45D 2034/007

(57) ABSTRACT

A functional jewelry configured to vitalize a fragrance is described. The functional jewelry includes a metal permeable sheet, an absorbable material and a shell defining an interior cavity. The absorbable material disposed in the interior cavity, the shell and the metal permeable sheet are separated structures. The upper part of the shell is set up with a holding rim that is congruent in shape and size with the metal permeable sheet. The metal permeable sheet is preferably composed of metal particles with spacing between them on the exterior surface. The use of the functional jewelry configured to vitalize a fragrance avoids the discomfort caused by direct contact of perfume with the human body. After absorbable material absorbs the fragrance, it enables the fragrance to volatilize uniformly and enduringly, over an extended period of time.

### TECHNICAL FIELD

In general, the present invention pertains to functional aspects of jewelry. In particular, the invention relates to functional jewelry configured to contain and gradually volatize fragrance as well as to systems and methods of producing the same.

### BACKGROUND OF THE INVENTION

There a variety of jewelry available nowadays, including high end jewelry, distinctive and fashionable jewelry. Nevertheless functional jewelry configured to receive, contain and gradually volatize fragrance, such as perfume, over a prolonged period of time, is much more seldom.

Typically perfumes are sprayed or otherwise applied to certain parts of the human body, directly contacted with human skin. Those who are allergic to direct contact with perfumes or have sensitive skin, however, can't use perfumes by applying them directly onto their skin, since a substantial amount of propellant included in perfume composition can be of harm to the human skin or cause discomfort to users.

Moreover since perfumes easily volatilize, for some users, applying perfume once a day doesn't meet their needs and requires additional application of the perfume from time to time throughout the day.

It is believed that the current state of the art is represented by the following patent literature: U.S. Pat. No. 2,620,227, U.S. Pat. No. 8,082,754, U.S. Pat. No. 7,178,363, U.S. Pat. No. 7,073,729, U.S. Pat. No. 7,896,256, US 20050034480, US 20130139549 and EP2229835.

### SUMMARY OF THE INVENTION

There a variety of jewelry available nowadays, including high end jewelry, distinctive and fashionable jewelry. Nevertheless functional jewelry configured to receive, contain and gradually volatize fragrance, such as perfume, over a prolonged period of time, is much more seldom.

Typically perfumes are sprayed or otherwise applied to certain parts of the human body, directly contacted with human skin. Those who are allergic to direct contact with perfumes or have sensitive skin, however, can't use perfumes by applying them directly onto their skin, since a substantial amount of propellant included in perfume composition can be of harm to the human skin or cause discomfort to users.

Moreover since perfumes easily volatilize, for some users, applying perfume once a day doesn't meet their needs and requires additional application of the perfume from time to time throughout the day.

It is believed that the current state of the art is represented by the following patent literature: U.S. Pat. No. 2,620,227, U.S. Pat. No. 8,082,754, U.S. Pat. No. 7,178,363, U.S. Pat. No. 7,073,729, U.S. Pat. No. 7,896,256, US 20050034480, US 20130139549 and EP 2229835.

### BRIEF DESCRIPTION OF THE DRAWINGS

The present invention will be understood and appreciated more comprehensively from the following detailed description taken in conjunction with the appended drawings in which:

FIG. 1 is an exploded view of exemplary functional jewelry in accordance with the present invention, embodying a pendant:

FIG. 2 is a longitudinal cross-sectional view of the pendant embodiment, shown in FIG. 1.

While the invention is susceptible to various modifications and alternative forms, specific embodiments thereof have been shown merely by way of example in the drawings. The drawings are not necessarily complete and components are not essentially to scale; emphasis instead being placed upon clearly illustrating the principles underlying the present invention.

### DETAILED DESCRIPTION OF THE INVENTION

Illustrative embodiments of the invention are described below. In the interest of clarity, not all features of actual implementation are described in this specification. It will of course be appreciated that in the development of any such actual embodiment, numerous implementation-specific decisions must be made to achieve the developers' specific goals, such as compliance with technology- or business-related constraints, which may vary from one implementation to another. Moreover, it will be appreciated that the effort of such a development might be complex and time-consuming, but would nevertheless be a routine undertaking for those of ordinary skill in the art having the benefit of this disclosure.

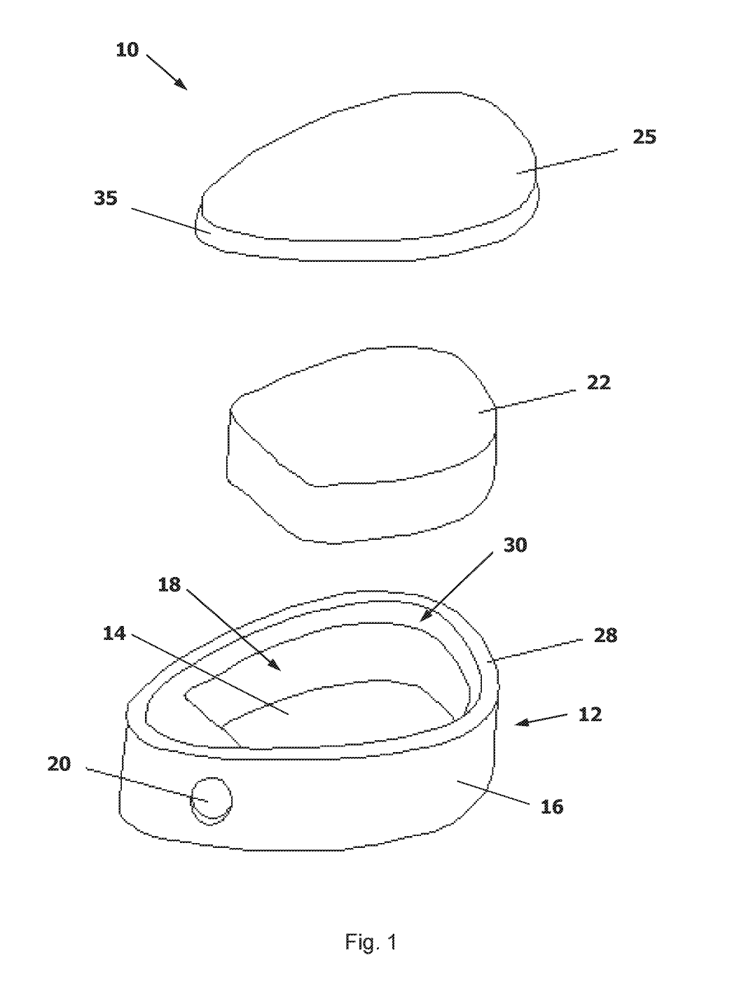

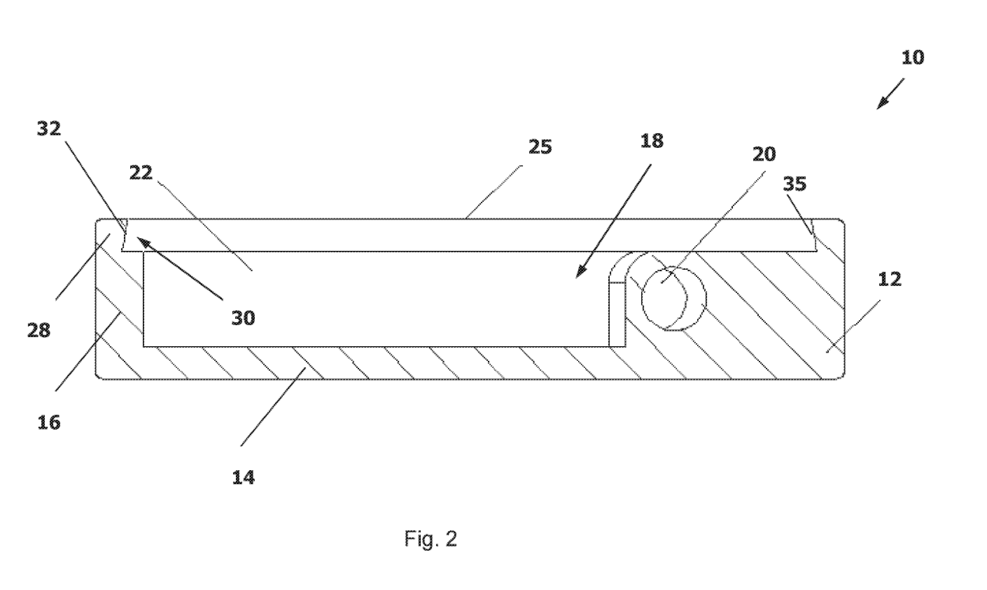

In accordance with some embodiments of the present invention, reference is now made to FIGS. 1 and 2, showing functional jewelry 10, embodied in a form of a pendant, configured to vitalize a fragrance. Functional jewelry 10 comprises shell 12. Shell 12 is made of stainless steel or other material possessing adequate aesthetic properties for jewelry. Shell 12 comprises bottom plate 14 and vertical sidewall 16, defining interior cavity 18 therein. Preferably the exterior shape of shell 12 is initially carved from a monolithic block of material that is shaped according to the appearance of functional jewelry 10 and then special purpose fixtures are used to process and form interior cavity 18 therein. Optionally through-hole 20 is defined by the apical portion of shell 12, rendering functional jewelry 10 suspendable as a pendant.

Shell 12 comprises interior cavity 18 used to accommodate at least one absorbent element 22, comprising an absorbing material configured to absorb frequents, such as perfumes, to extend the term of volatilization. Exemplary absorbing material of absorbent element 22 is a silicon gel. It should be acknowledged, however, that any polymeric compound, paper or other organic compounds or any other somewhat porous, membranous or fibrous material, characterized by a relatively large surface area that can efficiently absorb perfumes is equally applicable and contemplated in the context of absorbent element 22.

Functional jewelry 10 comprises sintered metal sheet 25, disposed on top of absorbent element 22 and embedded in top portion of shell 12. Sintered sheet 25 is preferably removable so that absorbent element 22 can be replaced to extend the service life functional jewelry 10. Sintered metal sheet 25 optionally comprises metallic particles of copper or other metal.

In some preferred embodiments, the top portion of vertical sidewall 16 of shell 12 comprises exterior rim 28, defining interior upper groove 30. Exterior rim 28 preferably comprises downwardly tapered inner face 32; whereas sintered metal sheet 25 preferably comprises upwardly tapered outer face 35. Functional jewelry 10 is preferably assembled by shrink fitting sintered metal sheet 25 within interior upper groove 30 defined by exterior rim 28 at the top portion of vertical sidewall 16. Shrink fitting, as referred to herein, includes increasing the temperature of shell 12 to about 150 degrees Celsius, thereby causing shell 12 to expand and assume somewhat lager dimensions, while decreasing the or at least maintaining the temperature of sintered metal sheet 25, for instance by freezing sintered metal sheet 25, so that sintered metal sheet 25 is introducible into interior upper groove 30 defined by exterior rim 28 at the top portion of vertical sidewall 16. Upon returning to a normal temperate, shell 12 shrinks down and effectively locks sintered metal sheet 25. Preferably upon locking sintered metal sheet 25 in place, downwardly tapered inner face 32 of exterior rim 28 surpasses upwardly tapered outer face 35 of sintered metal sheet 25 so that the former is disposed on top of the latter.

It is noted that the method of shrink fitting sintered metal sheet 25 in shell 12 allows affixing sintered metal sheet 25 in shell 12 so that the upper surface of sintered metal sheet 25 does not surpass the upper surface of vertical sidewall 16. In some preferred embodiments, exterior rim 28 is thicker than sintered metal sheet 25 so that the upper surface vertical sidewall 16 surpasses the upper surface of sintered metal sheet 25 by about 100 to 1500 micron.

Since sintered metal sheet 25 comprises metal particles, upper surface sintered metal sheet 25 forms numerous gaps, so that perfumes can spontaneously permeate through these gaps into absorbent element 22 of functional jewelry 10 in a liquid form and then to volatilize slowly and enduringly outside through these gaps in a gaseous form. Perfume, essence, sesame oil or other fragrances can be sprayed directly or otherwise applied onto sintered metal sheet 25, such as by dripping, soaking, filling or smearing.

Other connection techniques in lieu of shrink fitting between sintered metal sheet 25 and shell 12 also can be employed to make it easier to replace absorbent element 22 in interior cavity 18 of functional jewelry 10, such as snapping connection or buckle connection.

It should be acknowledged that embodiments of functional jewelry 10 are not limited to pendants but equally applicable to a key chain, bracelet etc.

It will be appreciated by persons skilled in the art that the present invention is not limited by what has been particularly shown and described herein above. Rather the scope of the invention is defined by the claims which follow:

What is claimed is:

1. A functional jewelry configured to volatize a fragrance, said functional jewelry comprises:
(a) a shell comprising:
(I) a bottom plate;
(II) a vertical sidewall, erecting essentially orthogonally to said bottom plate and forming a continuum therewith; wherein said sidewall defines an interior cavity in said shell;
(III) an exterior rim at a top portion of said sidewall, defining an interior upper groove;
(IV) a downwardly tapered inner face of said exterior rim at said top portion of said sidewall;
(b) an absorbent element, said absorbent element is introducible into said interior cavity of said shell; wherein said absorbent element comprises absorbing element configured to absorb said fragrance;
(c) a permeable metal sheet comprising:
(I) a shape essentially congruent to said interior upper groove;
(II) a plurality of metal particles defining a plurality of gaps therebetween;
(III) an upwardly tapered outer face;
wherein upon introducing said permeable metal sheet into said interior upper groove, said downwardly tapered inner face of said exterior rim is disposed on top of said upwardly tapered outer face of said permeable metal sheet; thereby essentially locking said permeable metal sheet in said interior upper groove.

2. The functional jewelry as set forth in claim 1, wherein said permeable metal sheet is affixed within said interior upper groove by shrink fitting.

3. The functional jewelry as set forth in claim 1, wherein said shell further comprises a portion defining a through-hole, rendering said jewelry suspendable.

4. The functional jewelry as set forth in claim 1, wherein an upper surface of said permeable metal sheet does not surpass an upper surface of said vertical sidewall.

5. The functional jewelry, as set forth in claim 4, wherein said upper surface of said vertical sidewall surpasses said upper surface of said permeable metal sheet by 100 to 1500 micron.

6. The functional jewelry as set forth in claim 1, wherein said absorbent element is replaceable.

7. The functional jewelry as set forth in claim 1, wherein said permeable metal sheet is removable.

8. The functional jewelry as set forth in claim 1, wherein gaps in said permeable metal sheet configured to sustain permeation of said fragrant through said gaps into said absorbent element in a liquid form upon application of said fragrant onto said permeable metal sheet and volatilization of said fragrant through said gaps in a gaseous form.

9. The functional jewelry, as set forth in claim 8, wherein said application is selected from the group consisting of: dripping, soaking, spraying or smearing.

10. The functional jewelry as set forth in claim 1, embodied in a form selected from the group consisting of: pendant, key chain and bracelet.

11. A method of manufacturing a functional jewelry configured to volatize a fragrance, said method comprises:
(a) providing a shell comprising:
(I) a bottom plate;
(II) a vertical sidewall, erecting essentially orthogonally to said bottom plate and forming a continuum therewith;
wherein said sidewall defines an interior cavity in said shell;
(b) forming an exterior rim at a top portion of said sidewall, defining an interior upper groove;
(c) shaping a downwardly tapered inner face of said exterior rim at said top portion of said sidewall;
(d) providing an absorbent element, wherein said absorbent element comprises absorbing element configured to absorb said fragrance;
(e) introducing said absorbent element is into said interior cavity of said shell;
(f) providing a permeable metal sheet comprising:
(I) a shape essentially congruent to said interior upper groove;
(II) a plurality of metal particles defining a plurality of gaps therebetween;
(g) forming an upwardly tapered outer face on said permeable metal sheet;
(h) introducing said permeable metal sheet into said interior upper groove;
(i) disposing said downwardly tapered inner face of said exterior rim on top of said upwardly tapered outer face of said permeable metal sheet; thereby essentially locking said permeable metal sheet within said interior upper groove.

12. The method of manufacturing a functional jewelry as set forth in claim 11, wherein said introducing is achieved by heating said shell and wherein said disposing is achieved by returning of said shell to a normal temperature.

13. The method of manufacturing a functional jewelry as set forth in claim 11, further comprises forming a through-hole in a portion of said shell, thereby rendering said jewelry suspendable.

14. The method of manufacturing a functional jewelry as set forth in claim 11, wherein an upper surface of said permeable metal sheet does not surpass an upper surface of said vertical sidewall.

15. The method of manufacturing a functional jewelry as set forth in claim 14, wherein said upper surface of said vertical sidewall surpasses said upper surface of said permeable metal sheet by 100 to 1500 micron.

16. The method of manufacturing a functional jewelry as set forth in claim 11, further comprises replacing said absorbent element.

17. The method of manufacturing a functional jewelry as set forth in claim 11, further comprises removing said permeable metal sheet.

18. The method of manufacturing a functional jewelry as set forth in claim 11, further comprises permeating said fragrant through said gaps into said absorbent element in a liquid form, upon applying said fragrant onto said permeable metal sheet and volatilizing said fragrant through said gaps in a gaseous form.

19. The method of manufacturing a functional jewelry as set forth in claim 18, wherein said applying is selected from the group consisting of: dripping, soaking, spraying or smearing.

20. The method of manufacturing a functional jewelry as set forth in claim 11, further comprises embodying said shell in a form selected from the group consisting of: pendant, key chain and bracelet.
